(19) United States
(12) Patent Application Publication
(10) Pub. No.: US 2016/0143756 A1
(43) Pub. Date: May 26, 2016

(54) ENDOVASCULAR MEDICAL DEVICE

(71) Applicant: Pegasus Therapeutics LLC, Lancaster, MA (US)

(72) Inventors: David A. Rezac, Westborough, MA (US); Aubry E. Shackelford, Lancaster, MA (US); Amos G. Cruz, Wrentham, MA (US); Jonathan B. O'Keefe, North Attleboro, MA (US); Jeffrey C. Cerier, Franklin, MA (US); Timothy W. Robinson, Sandown, NH (US)

(21) Appl. No.: 14/951,391

(22) Filed: Nov. 24, 2015

Related U.S. Application Data
(60) Provisional application No. 62/084,063, filed on Nov. 25, 2014.

(51) Int. Cl. A61F 2/88; A61F 2/90

(52) U.S. Cl. CPC A61F 2/88; A61F 2/90; A61F 2002/825

(57) ABSTRACT

An endovascular medical device that, when inserted into a vessel having one or more dilations and one or more narrowings in close proximity, redefines the vessel opening (lumen) along the longitudinal axis of the device, separates that lumen from the excess dilated volume within the dilation, and props open the lumen within the narrowing. The device is constructed in a manner to alter the hemodynamics at the interface between the redefined lumen and excess dilated volume, to facilitate the healing process within the dilated vessel. The device is constructed in a manner to provide sufficient radial force to open a narrowed portion of the vessel, to facilitate flow through the vessel. The device is constructed substantially from bioresorbable materials so as to eventually allow for the return of normal vasomechanics and cyclic wall stresses.

### CROSS-REFERENCE TO RELATED APPLICATIONS

This application claims the benefit of U.S. Provisional Application No. 62/084,063, filed Nov. 25, 2014, titled “Endovascular Medical Device,” the disclosure of which is hereby incorporated by reference in its entirety herein. Any and all priority claims identified in the Application Data Sheet, or any correction thereto, are hereby incorporated by reference under 37 CFR 1.57.

### FIELD

This disclosure pertains to medical devices. More specifically, the disclosure generally relates to an endovascular device for use within passages within the body having one or more dilations and one or more narrowings in close proximity.

### BACKGROUND

The body has multiple lumens which may experience persistent dilations, or enlargements greater than the normal lumen cross-sectional area. In the cardiovascular system, these persistent dilations are referred to as aneurysms or ectasia, and may have differing morphologies and etiologies. Within the lumen proximally and/or distally of these persistent dilations, there may also be a narrowing of the lumen to a smaller than normal cross-sectional area. In the cardiovascular system, these persistent narrowings are referred to as stenoses, and may also have differing morphologies and etiologies.

Many different types of interventional therapies exist for treating endovascular complications such as aneurysms and stenoses, including arterial stenosis interventional therapy, small-diameter arterial aneurysm interventional therapy, and large-diameter arterial aneurysm interventional therapy. However, conventional interventional therapies do not treat both stenoses and aneurysms simultaneously. In fact, complications can arise when two types of interventional therapies are needed. For example, arterial stenosis interventional therapies typically involve the use of stents, which are tubular prostheses capable of providing enough radial force to hold open the vessel. However, placement of a stent in a stenotic region near an aneurysmal region can result in the stent protruding into the aneurysmal region, which poses risks such as occlusion or embolization. In addition, the configuration of conventional stent structures does not provide sufficient diversion of blood flow to treat aneurysms. Treatment problems can also arise when conventional small-diameter arterial aneurysm interventional therapies and large-diameter arterial aneurysm interventional therapies are used where stenosis intervention is needed as well.

Generally, medical devices are made of long-lasting biocompatible materials (“biodurable”) which can result in issues that include, without limitation, long-term immune rejection, chronic inflammation, device mechanical failure, potential to limit future treatment options, or limited ability to increase in size in response to natural growth. Such issues may be particularly important to, without limitation, a pediatric demographic whose bodies continue to grow after the therapeutic time frame. Bioresorbable materials have been used in the construction of medical devices to address these issues. “Bioresorbable” is a term used in this application to indicate that a specific material and/or the majority of a device will eventually disappear after having been implanted.

Aneurysms and stenoses can occur next to each other. For example, Kawasaki Disease is an acute, self-limiting yet generalized systemic vasculitis of yet unknown etiology that occurs predominantly in infants and young children under five in which one or more aneurysms and stenoses can form next to each other. While it affects the entire cardiovascular system, the mortality and morbidity are predominantly associated with cardiac sequelae, primarily coronary artery ectasia and/or aneurysms that often progress to stenosis or occlusion while the children are still young. The current gold standard treatment in the event of this major adverse coronary event is the coronary artery bypass graft, which is a highly invasive and traumatic procedure. These cases demonstrate the need for a bioresorbable endovascular interventional therapy for the concurrent treatment of an aneurysm and a stenosis having close proximity within a small-diameter lumen.

### SUMMARY

The systems, methods, and devices discussed herein each have several aspects, no single one of which is solely responsible for its desirable attributes. Without limiting the scope of this invention as expressed by the claims which follow, some features are discussed briefly below. After considering this discussion, and particularly after reading the section entitled “Detailed Description,” one will understand the advantageous features of this device.

Generally, the present disclosure comprises a bioresorbable endovascular medical device for use within body passages having one or more zones of dilation, such as arterial aneurysms, and one or more zones of narrowing, such as arterial stenosis, within close proximity. The device is constructed, arranged and can be placed into a body passage so as to redefine the lumen. Within the zone of dilation, the device separates the excess dilated volume and promotes healing of the body passage within that volume. Within the zone of narrowing, the device imparts sufficient radial support to hold open the passage and maintain fluid patency through the lumen. The device is fabricated such that it disappears, at least in part, after a therapeutic time frame.

In one aspect, a bioresorbable endovascular medical device for placement into a diseased blood vessel that has a dilated portion adjacent a narrowed portion is disclosed. The medical device can include a generally tubular structure defining a longitudinal axis and a device lumen, the generally tubular structure having at least first and second parts spaced apart along the longitudinal axis, the at least first and second parts being discreet and yet integrally connected to one another and made of substantially the same material, the generally tubular structure configured for placement in a diseased blood vessel that has a dilated portion adjacent a narrowed portion, the blood vessel having a pre-disease healthy cross-sectional area that is smaller than a cross-sectional area of the dilated portion and larger than a cross-sectional area of the narrowed portion. The first part of the generally tubular structure can include a plurality of rings, each ring made up of a plurality of struts and each ring spaced apart from any adjacent ring of the plurality of rings along the longitudinal axis, and can include at least one connecting strut to connect adjacent rings of the plurality of rings, wherein when placed into the narrowed portion of the diseased blood vessel, the first part of the generally tubular structure is configured to increase the cross-sectional area of the narrowed portion and maintain an increased cross-sectional area. The second part of the generally tubular structure can include a tubular mesh, wherein when placed into the dilated portion of the diseased blood vessel, the tubular mesh is configured to direct blood flow along the device lumen while decreasing the flow into the dilated portion of the diseased blood vessel outside of the device lumen, the tubular mesh being porous to thereby promote the formation of thrombus within the dilated portion of the diseased blood vessel outside of the device lumen.

In another aspect, a bioresorbable endovascular medical device for placement into a diseased blood vessel having an aneurysm adjacent a stenosis is disclosed. The medical device can include a first generally tubular structure configured such that when placed into a stenosis in a diseased blood vessel, the first generally tubular structure serves to open the stenosis and maintain that opening, and can include a second generally tubular structure connected to the first generally tubular structure that, when placed into the aneurysm of the blood vessel, is configured to establish a vessel lumen and separate excess dilated volume from the vessel lumen.

In another aspect, a method of treating an aneurysm and a stenosis that are adjacent one another in a blood vessel is disclosed. The method can include advancing a delivery device with a bioresorbable endovascular medical device in a collapsed state to a treatment location in a diseased blood vessel, and can include deploying the bioresorbable endovascular medical device, the bioresorbable endovascular medical device comprising a generally tubular structure, a first portion of the generally tubular structure being within a stenosis and a second portion of the generally tubular structure being within an aneurysm.

Details of one or more implementations of the subject matter described in this specification are set forth in the accompanying drawings and the description below. Other features, aspects, and advantages will become apparent from the description, the drawings, and the claims. Note that the relative dimensions of the following figures may not be drawn to scale.

### BRIEF DESCRIPTION OF THE DRAWINGS

Various embodiments are depicted in the accompanying drawings for illustrative purposes, and should in no way be interpreted as limiting the scope of the inventions, in which like reference characters denote corresponding features consistently throughout similar embodiments.

FIG. 1 is a side view of an embodiment of a medical device of the present disclosure in its collapsed state on an exemplary delivery system.

FIG. 2 is a side view of a medical device of FIG. 1 in its expanded state, shown as if mounted on a solid cylinder to facilitate viewing.

FIG. 3 is a detail view of the connection between the aneurysm treatment and stenosis treatment zones of the medical device of FIG. 1.

FIG. 4A is a cross-sectional view from the radial direction of a blood vessel having an aneurysm, and a stenosis distal to the aneurysm.

FIG. 4B is a cross-sectional view from the radial direction of a blood vessel having an aneurysm, and a stenosis proximal and distal to the aneurysm.

FIG. 4C is a cross-sectional view from the longitudinal direction of a blood vessel having an aneurysm.

FIG. 5A is a side view of the delivery of a medical device of FIG. 1 in its collapsed state being delivered to and deployed within the lumen shown in FIGS. 4A.

FIG. 5B is a side view of the deployment of the stenosis treatment zone of a medical device of FIG. 1 by means of an inflated balloon.

FIG. 5C is a side view of the deployment of the aneurysm treatment zone of a medical device of FIG. 1 by means of self-expansion.

FIG. 5D is a side view of the final disposition of the medical device of FIG. 1 in its expanded state.

FIG. 6 is a side view of another embodiment of a medical device of the present disclosure in its expanded form, shown as if mounted on a solid cylinder to facilitate viewing.

FIG. 7 is a side view of another embodiment of a medical device of the present disclosure in its expanded form, shown as if mounted on a solid cylinder to facilitate viewing.

FIG. 8 is a cross-sectional view of elements of the medical device of Figure

FIG. 9 is a side view of another embodiment of a medical device of the present disclosure in its expanded form, shown as if mounted on a solid cylinder to facilitate viewing.

FIG. 10 is a side view of another embodiment of a medical device of the present disclosure in its expanded form, shown as if mounted on a solid cylinder to facilitate viewing.

FIG. 11 is a cross-section view of elements of the medical device of FIG. 10.

FIG. 12 is a side view of another embodiment of a medical device of the present disclosure in its expanded form.

FIG. 13 is a side view of the medical device of FIG. 12 deployed within a vessel having an aneurysm and two stenoses.

FIG. 14 is a side view of the medical device of FIG. 12 deployed within a vessel having only an aneurysm and with the stenosis treatment zones used to prevent migration.

### DETAILED DESCRIPTION

This disclosure can be accomplished in a medical device that is constructed and arranged to be placed into a blood vessel in the location of an aneurysm and a stenosis in close proximity. In the aneurysmal region, the device maintains a functional vessel lumen while also separating the lumen from the excess dilated volume. The device is sufficiently porous so as to promote gradual thrombosis within the aneurysm. In the stenotic region, the device maintains a functional vessel lumen while holding open the vessel to achieve a therapeutically targeted cross-sectional area. The device has a sufficient radial force so as to hold open the vessel to maintain a greater cross-sectional area and/or to impede progression of a stenosis, depending on the embodiment of the device and therapeutic targets. The device is constructed of bioresorbable materials, at least in part, that disappear after a therapeutic time frame.

The following description and the accompanying drawings describe and illustrate several embodiments of the medical device; however, these are exemplary and not limiting as the device could be constructed and arranged differently. Also, features in one embodiment can be used in other embodiments.

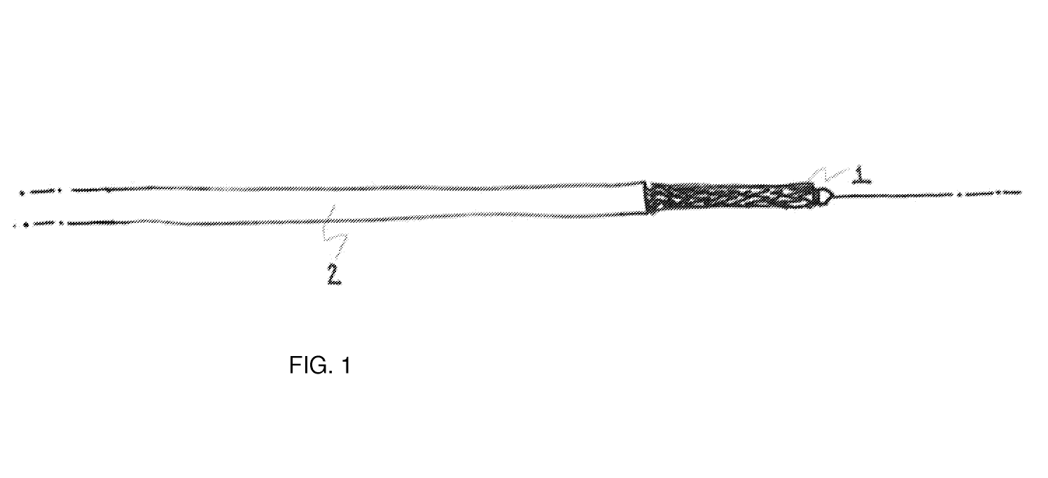

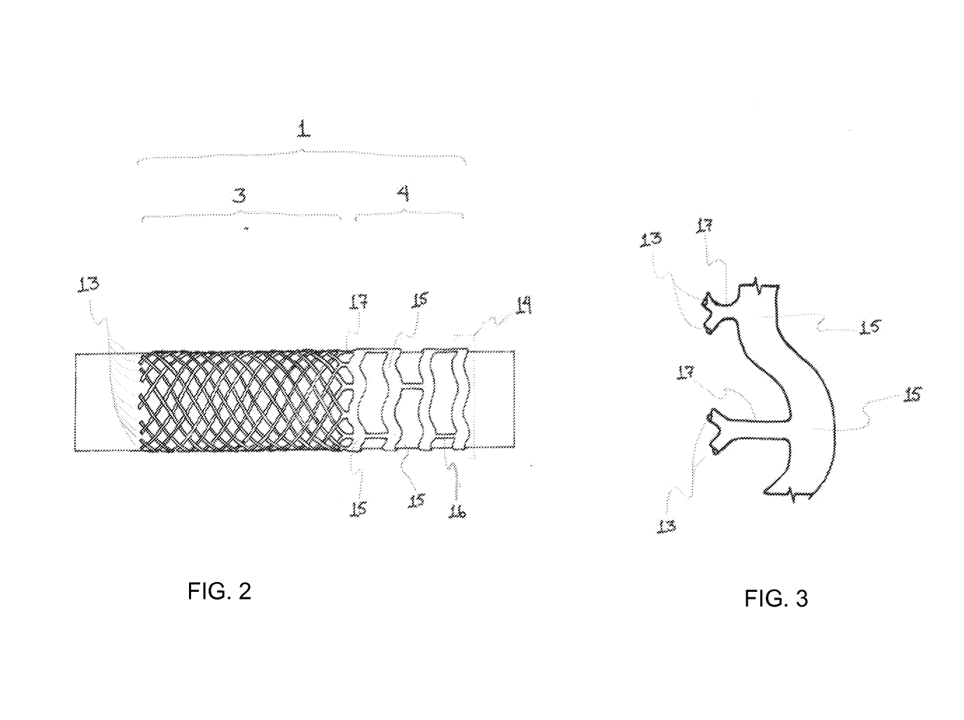

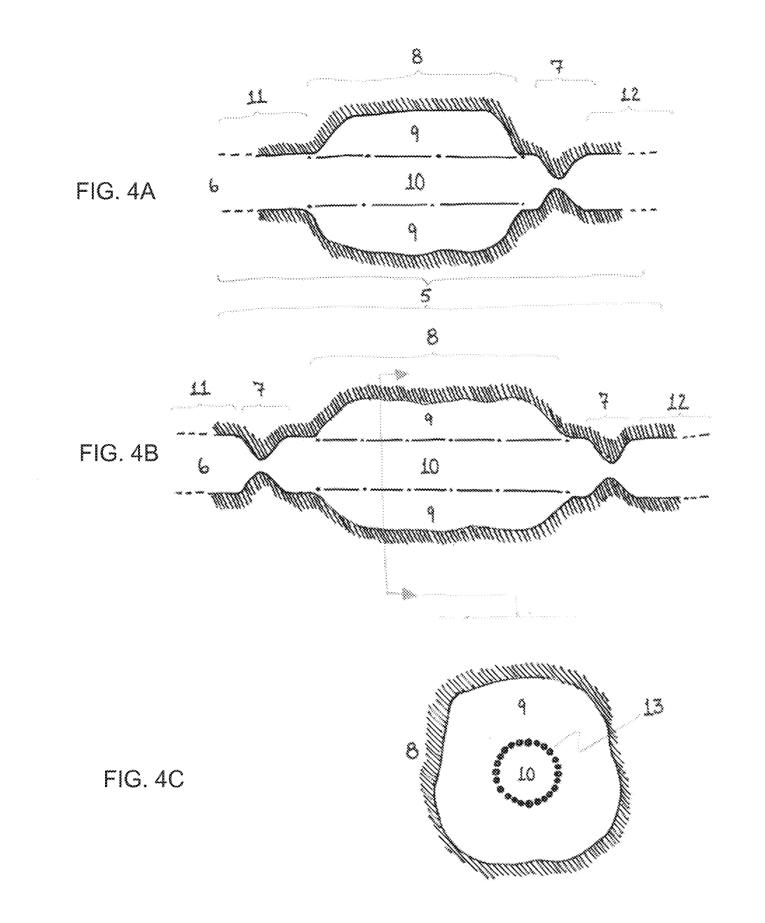

Referring now to the disclosure in more detail, in FIG. 1 to FIGS. 5A-5D there is shown one non-limiting embodiment of a medical device 1 of the present disclosure. FIG. 1 is a side view of an embodiment of a medical device of the present disclosure in its collapsed state on an exemplary delivery system, and FIG. 2 is a side view of the medical device shown in FIG. 1 in its expanded state. In FIG. 2, the frame (also referred to as a tubular structure) of the medical device is straight, but the skilled artisan will appreciate that the frame of the medical device can be angled and/or curved in accordance with blood vessel morphology, and that the frame of the medical device can be angled and/or curved to facilitate treatment of diseased regions of blood vessel morphology. For example, in some embodiments, the frame of the medical device is straight, and in some embodiments, the frame of the medical device is straight, angled, and/or curved. FIG. 3 is a detail view of an embodiment of transition element 17 that can exist at the interface of the aneurysm treatment longitudinal zone 3 and the stenosis treatment longitudinal zone 4 in an embodiment of the medical device 1. In some blood vessel morphologies, the blood vessel can be diseased and have one or more aneurysms and one or more stenoses in close proximity. For example, FIGS. 4A-4C illustrate cross-sectional views of a diseased blood vessel having one or more aneurysms and one or more stenoses in close proximity.

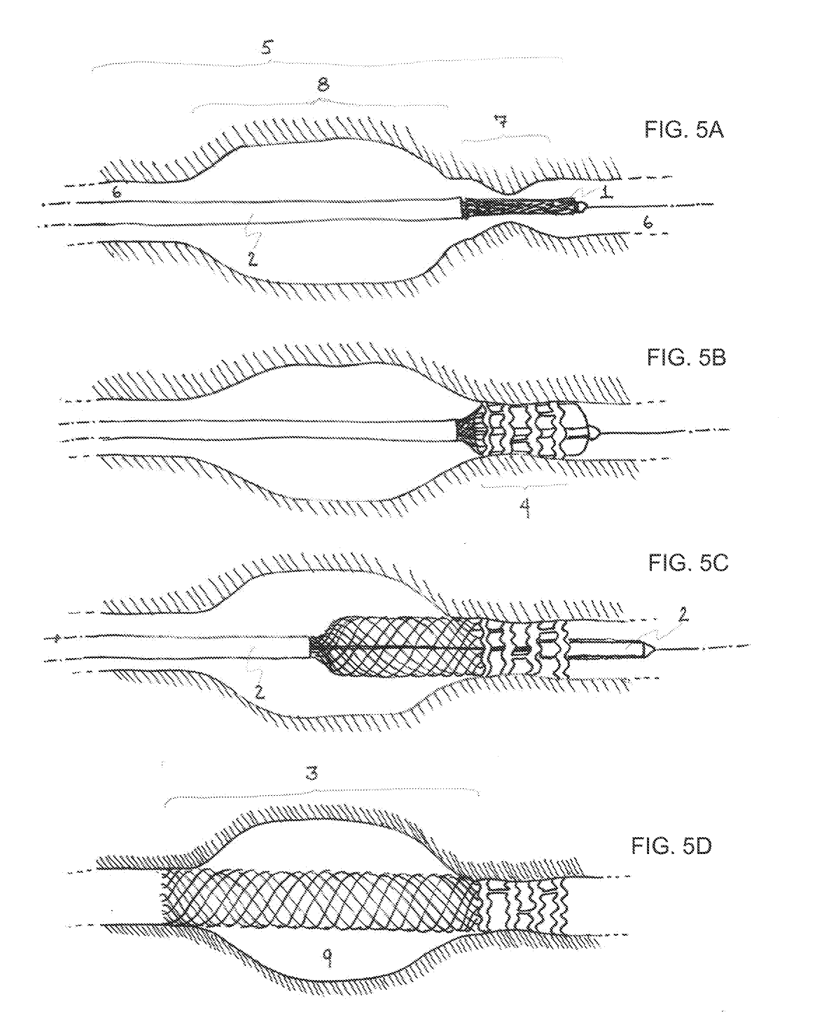

In some embodiments, the medical device 1 can be delivered to a diseased vessel 5 by an exemplary delivery device 2 as shown in FIGS. 5A-5D. In some embodiments, the diseased vessel 5 can have a stenotic lesion 7 and an aneurysmal lesion 8 in close proximity, having no more than about 50 mm of non-diseased vessel between the lesions. Generally, there is no more than about 20 mm of non-diseased vessel between the lesions, but the skilled artisan will appreciate that medical device 1 can be adapted to accommodate and treat lesions separated by any distance dictated by patient needs, such as, for example, separations less than 20 mm, between 20 mm and 50 mm, and greater than 50 mm. For example, FIGS. 5A-5D are a series of side views of the medical device 1 of FIGS. 1 and 2 being delivered to and deployed within a lumen of a diseased blood vessel having lesions in close proximity. In particular, FIG. 5A shows the medical device 1 being delivered to a diseased vessel in a collapsed state, FIG. 5B shows the medical device 1 being deployed to a stenosis lesion 7 of the diseased vessel, FIG. 5C shows the medical device 1 being deployed to an aneurysmal lesion 8 of the diseased vessel, and FIG. 5D shows a final disposition of the medical device 1 in its expanded state in the diseased vessel. In some embodiments, the medical device 1 can be deployed to the stenosis lesion 7 by means of an inflated balloon, and in some embodiments, the medical device 1 can be deployed to the aneurysmal lesion 8 by means of self-expansion.

As shown in FIG. 2, the medical device 1 can have an aneurysm treatment longitudinal zone 3 and a stenosis treatment longitudinal zone 4, which can advantageously be used to treat anuerysmal lesions and stenosis lesions, respectively. In some embodiments, the medical device 1 can exist in a collapsed and/or an expanded state. In its expanded state, the medical device 1 can have a diameter less than about 6 mm. For example, in some embodiments, the expanded diameter can be between 1 mm and 5 mm, and in some embodiments, the expanded diameter can be between 2 mm and 4 mm. In its expanded state, the medical device 1 can have a length less than about 150 mm. For example, in some embodiments, the expanded length can be between 10 mm and 80 mm. The individual lengths of the aneurysm treatment longitudinal zone 3 and a stenosis treatment longitudinal zone 4 can be more than 5 mm each, although any suitable individual length is appreciated. Further, in some embodiments, the length of the delivery device 2 can be greater than about 50 cm, and in some embodiments, the length of the delivery device 2 can be greater than about 110 cm, although any suitable length of the delivery device 2 is appreciated.

In more detail, still referring to the embodiment disclosed in FIG. 1 to FIGS. 5A-5D, when inserted into the lumen 6 of a diseased vessel 5, the medical device 1 redefines the vessel opening (redefined lumen 10) along its longitudinal axis. Within the aneurysmal lesion 8, the medical device 1 partially or wholly separates that redefined lumen 10 from the excess dilated volume 9 by means of creating distinct zones of fluid flow inside of and outside of (with respect to the radial direction) the aneurysm treatment longitudinal zone 3. Within the stenotic lesion 7, the medical device 1 provides sufficient radial force to hold open the vessel and allow sufficient flow through the stenosis treatment longitudinal zone 4. The medical device 1 provides the necessary mechanical characteristics for an endovascular medical device to redefine and maintain an open lumen by providing a hemodynamically stable channel through which a substantial portion of the incoming fluid can flow under normal physiological conditions. The aneurysm treatment longitudinal zone 3 provides the necessary characteristics for an endovascular medical device to prevent or allow specific proportions of the incoming fluid to pass from the lumen to the dilated volume. These characteristics include having an effective porosity, which ranges from minimal porosity of 0% of the surface area for cases where fast thrombosis within the aneurysm is required to relieve pressure, to sufficient porosity up to approximately 90% of the surface area to alter the flow characteristics enough to promote gradual thrombosis within the aneurysm. The porosity can be between 0% and about 10%, or can be between about 10% and about 40%, or can be between about 40% and about 60%, or can be between about 60% and 90%, or the like, such as any suitable porosity. In some embodiments, the porosity can between about 30% and about 70%. The porosity can be established by, without limitation, the properties of the material used having an inherent porosity, the physical addition of pores through the layer, the interstitial spacing between elements comprising the layer containing the aneurysm treatment longitudinal zone 3, or interaction between layers. The stenosis treatment longitudinal zone 4 provides the necessary characteristics for an endovascular medical device to initially maintain a lumen at a specified cross-sectional area. These characteristics include having sufficient radial force to open the narrowing and limit recoil.

The deployment characteristics of the medical device 1 can be self-expanding so that it will change from a collapsed state to an expanded state by virtue of its own stored energy and/or interaction within the body, assist-expanding so that the change from a collapsed state to an expanded state requires additional energy supplied by the delivery device 2, or some combination thereof. The delivery device 2 allows the medical device 1 to be inserted and deployed into the diseased vessel 5 depending on the deployment characteristics, such as catheter delivery deployment by removing a sheath over the collapsed state of the medical device 1 having self-expanding characteristics, or insertion of a medical device 1 having its collapsed state surrounding a balloon which is subsequently inflated in a manner similar to that known within the art for the deployment of coronary, peripheral, self-expanding and balloon-expandable stents and vascular grafts.

The total thickness of medical device 1 can be controlled such that the redefined lumen has a mean cross-sectional area that is approximately equal to any of the cross-sectional areas of the proximal portion of the diseased vessel 11, distal portion of the diseased vessel 12, or theoretically normal vessel. Of course, any suitable thickness of medical device 1 and any suitable redefined cross-sectional area of medical device 1 is appreciated and envisioned. For example, in some embodiments, the redefined lumen can have a cross-sectional area that is within plus 50% or minus 30% of that of the un-dilated vessel adjacent to the dilation. The morphology of the vessel can include without limitation kinking, coiling, and/or tortuosity; the morphology of the stenotic lesion 7 can include without limitation asymmetrical distributions of deposits and/or tissues; the morphology of the aneurysmal lesion 8 can include without limitation saccular or fusiform characteristics. The morphology of the diseased vessel 5 need not be a uniformly cylindrical vessel with hourglass-shaped proximal and distal stenotic lesion 7 zones and a fusiform dilation aneurysmal lesion 8 zone as shown in FIGS. 4A-4C and FIGS. 5A-5D.

The construction details of medical device 1 as shown in FIG. 1 to FIGS. 5A-5D are that the medical device 1 is comprised of at least two discreet sections, the aneurysm treatment longitudinal zone 3 and the stenosis treatment longitudinal zone 4, and are constructed of bioresorbable synthetic polymers, including without limitation poly(glycolic acid) (“PGA”), poly(lactic acid) (“PLA”), polycaprolactone, poly(DTE carbonate), polydioxanone, poly(hydroxybutyrate), or poly(hydroxyvalerate). The structure of medical device 1 can be created by means of solid freeform fabrication, also referred to as additive manufacturing, using techniques known in the art of additive manufacturing. In some embodiments, the aneurysm treatment longitudinal zone 3 can be constructed as a braided mesh structure of a plurality of filaments 13, more than about six and less than about forty-eight, some of which are helically wound in one direction and some of which are helically wound in the opposite direction. In some embodiments, the stenosis treatment longitudinal zone 4 can be constructed as an assembly of one or more rings 14. For example, in some embodiments, the rings 14 can be undulating rings. In some embodiments, the stenosis treatment longitudinal zone 4 can be constructed as an assembly of one or more rings 14, each of which is comprised of a plurality of struts 15 situated at offset angles in the collapsed state between neighboring struts 15 to create an undulating construct, with a plurality of connecting elements 16 situated longitudinally between the rings 14. The number of rings 14 of the stenosis treatment longitudinal zone 4 can be dependent on the target therapeutic length of the stenosis treatment area. For example, in some embodiments, the number of rings 14 of the stenosis treatment longitudinal zone 4 can be four or more, although any suitable number of rings 14 is appreciated. Similarly, the number of struts 15 can be dependent on the target therapeutic diameter of the stenosis treatment area. For example, in some embodiments, is the number of struts 15 can be six or more, although any suitable number of struts 15 is appreciated. In some embodiments, the number of struts 15 can be between twelve and twenty-four. The number of connecting elements 16 between the rings 14 is at least one. For example, in some embodiments, the number of connecting elements 16 can be between three and six. In some embodiments, for each of the filaments 13 and rings 14, transition elements 17 can exist at the interface of the aneurysm treatment longitudinal zone 3 and the stenosis treatment longitudinal zone 4 to connect each of the filaments 13 to each of the rings 14, which are shown in more detail in FIG. 3. In some embodiments, transition elements 17 connect each of the filaments 13 to the first ring 14 proximally adjacent the aneurysm treatment longitudinal zone 3.

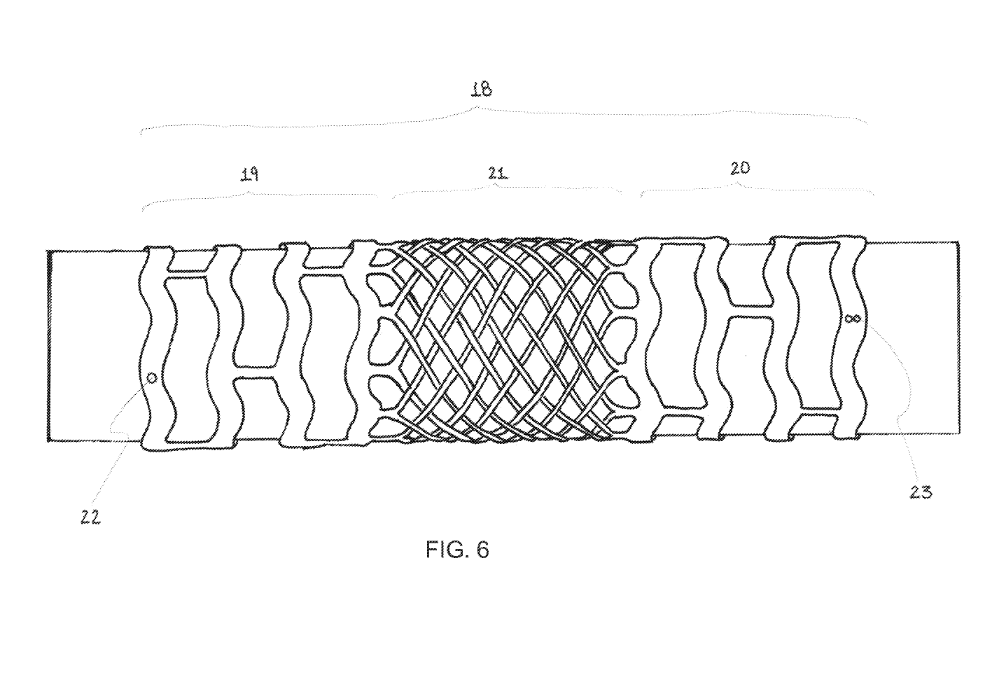

The subject medical device need not be limited to having only one of each treatment zone. For example, referring now to FIG. 6, the medical device 18 can include a proximal stenosis treatment longitudinal zone 19, a distal stenosis treatment longitudinal zone 20, an aneurysm treatment longitudinal zone 21 spanning therebetween, an optional proximal radiopaque marker 22, and an optional distal radiopaque marker 23. The physical characteristics of each zone, including without limitation the distance from center, the thickness, the surface features, and the profile, can change along the longitudinal and radial axes depending on the embodiment of the disclosure. In some embodiments, the proximal stenosis treatment longitudinal zone 19 can have a larger expanded diameter than the distal stenosis treatment longitudinal zone 20, advantageously reproducing a typical arterial blood vessel taper. The optional proximal radiopaque marker 22 and/or the optional distal radiopaque marker 23 each allow for viewing of the position of the medical device 18 during deployment, or some time thereafter, e.g., by means of fluoroscopy.

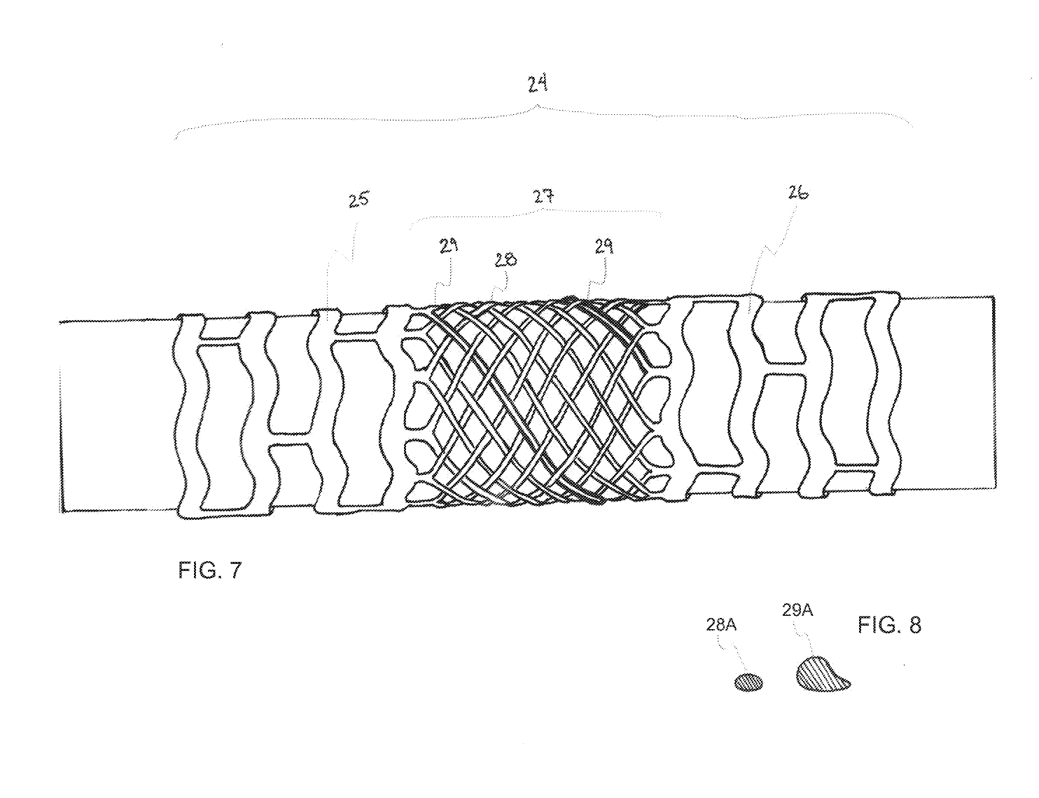

The subject medical device can have specific surface modifications, can be imbued with additional agents, and can be constructed of any bioresorbable materials. Referring now to an embodiment of the device shown in FIG. 7 to FIG. 8, the medical device 24 can include a proximal stenosis treatment longitudinal zone 25, a distal stenosis treatment longitudinal zone 26, and an aneurysm treatment longitudinal zone 27. In more detail, still referring to the embodiment of the device shown in FIGS. 7 and 8, the aneurysm treatment longitudinal zone 27 can consist of a braided mesh of multiple typical filament elements 28 having a cross-sectional profiles 28a and at least one atypical filament element 29 having a specifically modified cross-sectional profile 29a. The atypical filament element having a specifically modified cross-sectional profile 29a can be designed to induce helical flow within the redefined lumen. It should be noted that multiple atypical filament elements of this type can be incorporated into the device. The bioresorbable materials that the subject medical device can be made include metals, including without limitation magnesium, iron, or calcium; ceramics, including without limitation hydroxylapatite; synthetic polymers, including without limitation poly(glycolic acid) (“PGA”), poly(lactic acid) (“PLA”), polycaprolactone, poly(DTE carbonate), polydioxanone, poly(hydroxybutyrate), or poly(hydroxyvalerate); natural polymers, including without limitation collagen, elastin, fibrin, silk, and chitosan; or hydrogels and/or elastomers comprised of synthetic and/or natural polymers. These materials can be combined or hybridized to form a component of the device. These materials can have additional agents, such as pharmaceuticals, including without limitation sirolimus or heparin that have specific indications for use, or natural bio-active materials, including without limitation growth factors or cell adhesion peptide sequences, integrated into the material or on the surface of the material. These additional agents can be chemically selective such that they attract specific compounds, materials, cell types, or fluid components from surrounding tissues to generate a preferred concentration and mixture of host cells that promote healing along the dilated vessel segment. The materials can have specific surface preparations to enhance their functionality to achieve a variety of desired interactions with surrounding device elements and/or body tissues and fluids in a localized, oriented, selective, and/or tunable fashion; and the surface preparations can vary with respect to radial or longitudinal axes.

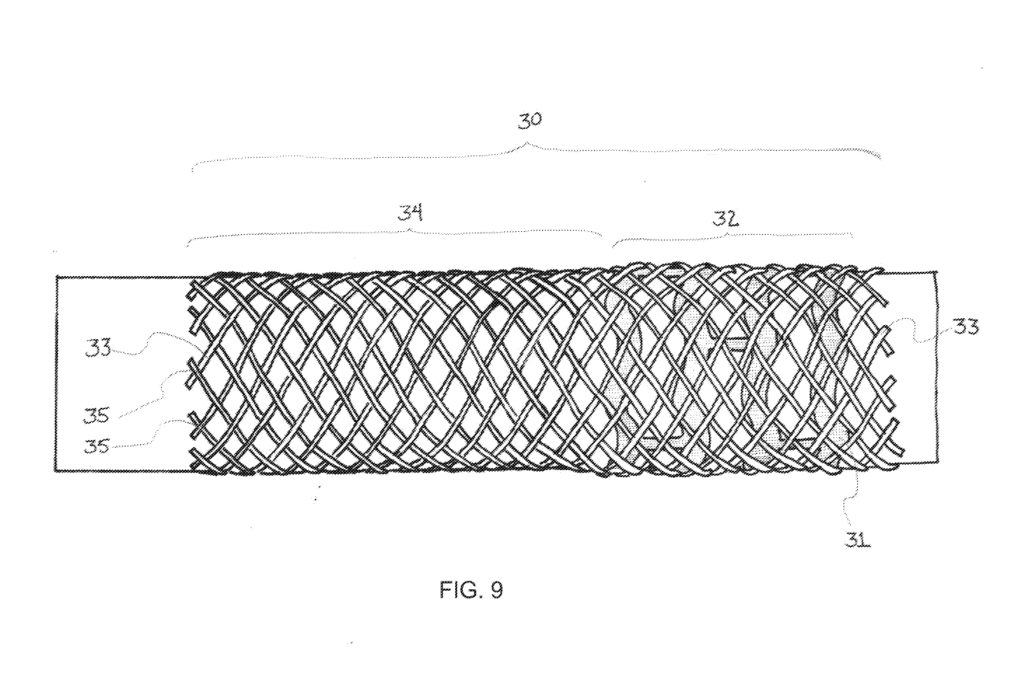

The subject medical device need not be limited to having only one integrated layer, and can include multiple layers to achieve the design. Referring now to an embodiment of the device shown in FIG. 9, the medical device 30 can include a structural layer 31 comprising part of a stenosis treatment longitudinal zone 32, a membrane layer 33 comprising part of an aneurysm treatment longitudinal zone 34, and an outer layer 35 having a separate outer coating of a mixture of collagen and elastin added to the membrane layer 33. The structural layer 31 and membrane layer 33 can be positioned with the structural layer 31 inside the membrane layer 33 as shown in FIG. 9, or vice versa, or can be constructed in a manner that allows one layer to perform the function of both the structural layer 31 and the membrane layer 33. The physical characteristics of each radial layer, including without limitation the distance from center, the thickness, the surface features, and the profile, can change along the longitudinal and radial axes depending on the embodiment of the disclosure. Each radial layer can be discontinuous along the longitudinal axis, and can be present in only specific zones. The length of each radial layer can be distinct relative to any adjacent layer. Furthermore, the radial layers can be constructed by means of, without limitation, forming a profiled layer of electrospun fibers; fiber bonding; solvent casting and particulate leaching; superstructure engineering; compression molding; extrusion; freeze-drying; phase separation; gas foaming; supercritical fluid processing; solid freeform fabrication; braiding or knitting filaments (fibers or wires) into a mesh; cutting tubing; injecting materials into a mold; depositing materials onto a surface; injecting material in situ; injecting material in situ and curing that material; wrapping filaments in concentric rings, coils, or helical wraps; or attaching two ends of a sheet of film together, or any other materials or fabrication techniques now known or developed in the future. The radial layers can be attached to one another, but need not be.

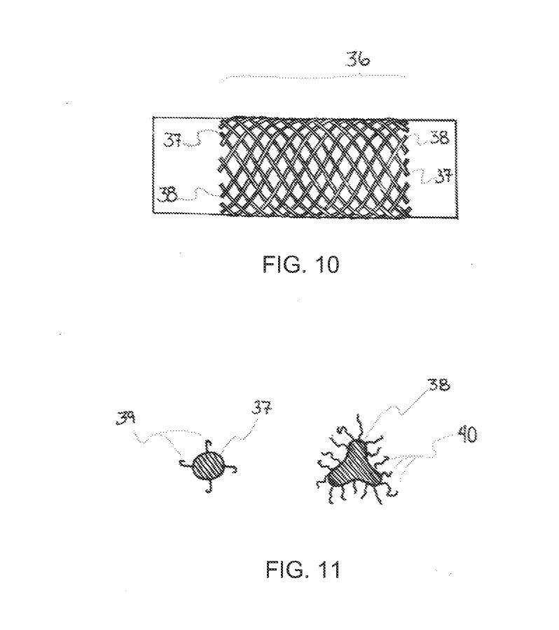

The subject medical device can achieve the desired characteristics for the treatment of an aneurysm or a stenosis by means of a localized modification of a given layer within the longitudinal treatment zone. Referring now to an embodiment of the device shown in FIG. 10 and FIG. 11, medical device 36 can include a single layer comprised of a braided mesh of barbed filaments 37 and fuzzy filaments 38. In more detail, still referring to the embodiment of the disclosure shown in FIG. 10 and FIG. 11, the device can be constructed of barbed filaments 37 made of a bioresorbable material such as PLA and fuzzy filaments 38 made of a bioresorbable material such as PLA into a mesh having a porosity defined by the diamonds formed by the spacing between the braided filaments. The barbed filaments 37 can have a circular profile and can have a surface treatment that creates micro-sized hooks 39 on the surface of the filament. Of course, any suitable shape and surface treatment is envisioned. The fuzzy filaments 38 can have a surface treatment that creates micro-sized yarns 40 on the surface of the filament. When expanded, the micro-sized hooks 39 and micro-sized yarns 40 can catch one another, providing for a mechanism to lock the device in its expanded state. In the medical device 36 shown in FIGS. 10 and 11, the filaments can be selectively augmented to achieve a coordinated interaction prior to, during, or after implantation. In some embodiments, an arrangement of material properties, geometry, or surface characteristics can be employed to create distinct interaction behavior between crossing members of a given structure. This interaction can be leveraged to a variety of ends, but can specifically be used to reinforce or “lock” an implant in the open state. In the event a balloon catheter is used to post-dilate and seat the device, this concept can improve the device's ability to resist recoil of a vessel segment. This behavior can be configured for permanence or transiency (for example, through the inclusion of micro-structures that acutely augment mechanical performance, but are quickly absorbed by the body such that they are only present and active for a portion of the device's overall therapeutic time frame).

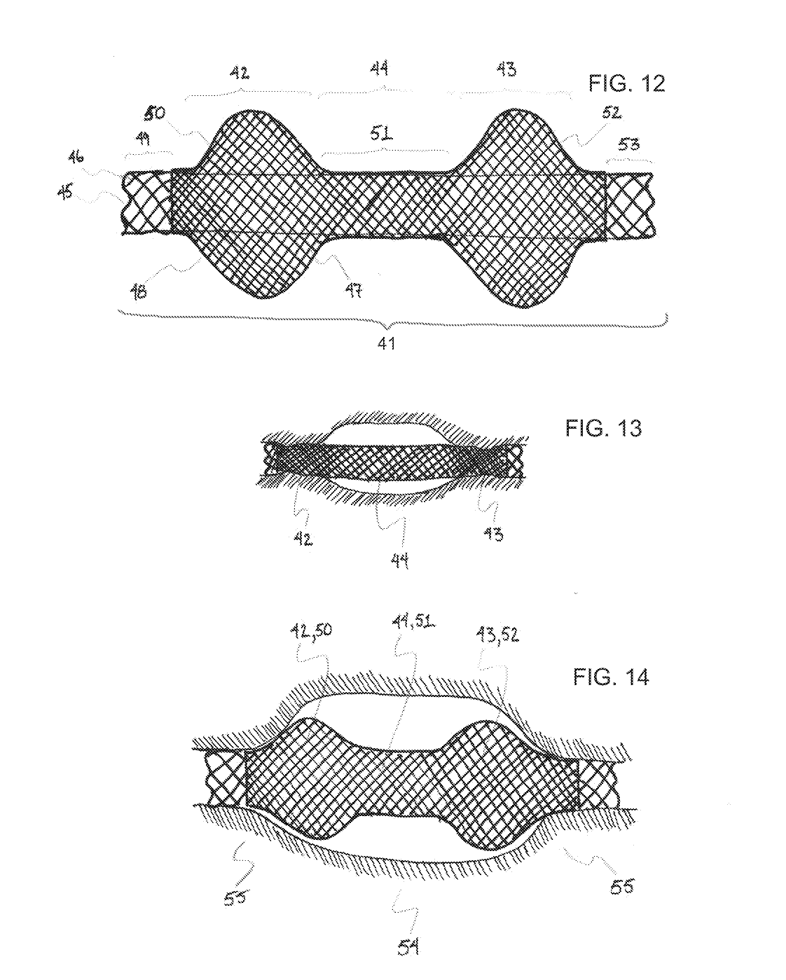

Referring now to an embodiment of the device shown in FIG. 12 to FIG. 14, medical device 41 can include a proximal stenosis treatment longitudinal zone 42, a distal stenosis treatment longitudinal zone 43, and an aneurysm treatment longitudinal zone 44. In some embodiments, the medical device can also include an inner layer 45, a membrane layer 46, a structural layer 47, and an outer layer 48. The membrane layer 46 can be constructed by means of attaching two ends of a thin sheet of PLA together into a concentric tube and drilling holes into that film. The inner layer 45 can be formed by application of a pharmaceutical agent to the membrane layer 46. The structural layer 47 can include a proximal non-protruding zone 49, a proximal protruding zone 50, a dilation bridging zone 51, a distal protruding zone 52, and a distal non-protruding zone 53. The membrane layer 46 can be attached to the structural layer 47 at the proximal non-protruding zone 49, dilation bridging zone 51, and distal non-protruding zone 53. The structural layer 47 can be constructed by braiding magnesium wire into a mesh and altering the lengths and locations of overlapping points so as to form, as an integral part of the structural layer 47, the proximal stenosis treatment longitudinal zone 42 and distal stenosis treatment longitudinal zone 43, which can comprise non-cylindrical shapes that protrude radially away from the redefined lumen formed by the membrane layer 46. The outer layer 48 can be formed by application of a PGA polymer coating on the magnesium wire surface. The protrusion geometry of the stenosis treatment zones provides additional radial forces that can be needed to hold open a stenosis.

Furthermore, it should be recognized that radial forces required to open a narrowed passage can also be used to anchor the device within the lumen without the presence of a stenosis, thus also providing a solution to issues related to the migration of a device in vivo. For example, as shown in FIG. 14, the protrusion geometry of the proximal protruding zone 50 and distal protruding zone 52 of the structural layer 47 can interact atraumatically with the wall of a vascular aneurysm to be aligned and selectively compressed by the aneurysm 54, aneurysm neck 55, and parent vessel geometries. The non-cylindrical protrusion can register to and become fixated against the aneurysm neck 55 in a manner that provides axial stability. Additionally, thrombus formation and endothelial ingrowth between the structural layer 47 and membrane layer 46 would further integrate the medical device 41 with surrounding biological tissues. It should be noted that the embodiment of a braid is shown for purposes of illustration and not limitation, meaning that any method of fabrication capable of achieving a non-cylindrical, selectively compressible structure suitable for interaction with the aneurysm wall is envisioned. Once delivered, the device would integrate with the vessel wall, providing structural support for the potentially weakened aneurysmal segment of the artery and attracting biological response consistent with the chemistry of the implanted material. Furthermore, taking advantage of the enhanced integration with biological tissues afforded by this embodiment, the outer layer can be coated, impregnated, or otherwise hybridized to deliver biologically influential agents to the site of the aneurysm that aid in tissue response and vascular remodeling. Such agents, for example, can include drugs, growth factors, stem cells, and other compounds known within the art of tissue engineering to have desirable biological effects within the context of aneurysmal disease.

In any of the embodiments described herein, the frame (also referred to as a tubular structure) of the medical device can be straight, angled, and/or curved in accordance with blood vessel morphology, and/or can be straight, angled, and/or curved to facilitate treatment of diseased regions of blood vessel morphology. For example, in some embodiments, the frame of the medical device is straight, and in some embodiments, the frame of the medical device is straight, angled, and/or curved.

To deliver and deploy the medical device described herein, common percutaneous interventional techniques can be used. In some cases, the lesions can be imaged prior to the procedure as part of an ongoing surveillance program, and other preparatory steps such as determining target therapeutic diameter and lengths or selection of appropriate percutaneous access points can be performed. Access to the arterial tree can be obtained with a puncture through the skin and introduction of a guidewire by standard Seldinger technique, usually to a femoral artery, but in some cases via alternative access sites such as to a brachial or radial artery. Access can be maintained for the delivery and deployment and can involve an introducer sheath or dilator. Once access is achieved, a guiding catheter can be introduced at the access point and routed to the approximate location of the lesions, and the lesions can be imaged, for example using fluoroscopy in conjunction with the injection of a radio-opaque dye through the guiding catheter, for multiple purposes including final selection of the target therapeutic sizing of the medical device. Other procedures, such as the delivery of pharmaceutical agents like heparin, can occur during this process. In some cases, a guide wire can be advanced past the location of the lesions, providing a direct route to the lesions. Preparation of the lesions can be performed using procedures such as angioplasty in which a balloon mounted on a hollow catheter is inserted over the guide wire, which then occupies a hollow lumen of the catheter. For example, using angioplasty procedures, the balloon can be pushed to the location of the stenotic lesion, then inflated to open the narrowing, then deflated, and then pulled back with the catheter to remove the balloon. The medical device described herein, being mounted in its collapsed state as part of a catheter-based delivery system, can be delivered to the location of the lesions over the guide wire and deployed. The actual method of deployment is dependent on the embodiment of the medical device described herein, as is described in more detail below. Once deployed in its expanded state, the delivery system can be withdrawn back over the guide wire. Post-deployment procedures, such as angiographic imaging, delivery of pharmaceutical agents, or angioplasty as described above, can occur after the deployment of the medical device. The guide wire and guiding catheter can then be removed and the access site can be closed and managed.

For example, FIGS. 5A-5D illustrate the steps involved in the delivery of the medical device to and deployment within a diseased vessel having an aneurysm and a stenosis according to some embodiments. In some embodiments, the medical device 1 can be mounted in its collapsed state on an exemplary delivery device 2, which can have an inner catheter, a balloon mounted at the distal end of the inner catheter and folded in its collapsed state, and the stenosis treatment longitudinal zone 4 in its collapsed state over the balloon, which can be crimped into place on the balloon. Furthermore, in some embodiments, the exemplary delivery device 2 can have an outer sheath, and the aneurysm treatment longitudinal zone 3 can be inserted between the outer sheath and the inner catheter, which can be achieved by stretching the aneurysm treatment longitudinal zone 3 in the longitudinal direction, which increases the length and reduces the inner diameter of the cylindrical form. In some embodiments, as shown in FIGS. 5A-5D, the stenosis treatment longitudinal zone 4 can be expanded from its collapsed state to its expanded state with assistance from the expansion of the balloon, and the aneurysm treatment longitudinal zone 3 can be self-expanding. The inner catheter of the delivery device 2 can be inserted over the guide wire, the guide wire then occupying a lumen within the inner catheter. The medical device 1 can then be pushed to the location of the lesions of the diseased vessel 5 by feeding the delivery device 2 over the guide wire. The medical device 1 is in the appropriate location with the stenosis treatment longitudinal zone 4 in approximate alignment with the stenotic lesion 7, as shown in FIG. 5A. Once the medical device 1 is in the appropriate location, the balloon mounted on the distal end of the inner catheter can be inflated, causing the stenosis treatment longitudinal zone 4 to move to its expanded state, as shown in FIG. 5B. The balloon can then be deflated into its collapsed state. The outer sheath can then be withdrawn while the inner cather may be pushed in, allowing the aneurysm treatment longitudinal zone 3 to self expand into the aneurysmal lesion 8, as shown in FIG. 5C. Once the medical device 1 has been fully deployed within the lumen 6 of a diseased vessel 5, the delivery device 2 can be removed, as shown in FIG. 5D. The medical device 1 redefines the vessel opening (redefined lumen 10) along its longitudinal axis. Within the aneurysmal lesion 8, the medical device 1 partially or wholly separates that redefined lumen 10 from the excess dilated volume 9 by means of creating distinct zones of fluid flow inside of and outside of (with respect to the radial direction) the aneurysm treatment longitudinal zone 3. Within the stenotic lesion 7, the medical device 1 provides sufficient radial force to hold open the vessel and allow sufficient flow through the stenosis treatment longitudinal zone 4.

It should be noted that device orientation and procedural sequence can be modified to accommodate various disease morphologies. For example, a stenotic lesion 7 located proximal to an aneurysmal lesion 8 may be treated using substantially the same methods. In addition, the sequence described above can be used to treat a stenotic lesion 7 located proximal or distal to an aneurysmal lesion 8, with subsequent treatment of the unaddressed stenotic lesion 7 by means of further installing a stenosis treatment longitudinal zone 32 within the stenotic lesion 7 through expansion of a balloon as previously described. Furthermore, aneurysmal lesions in close proximity to two or more stenotic lesions (such as the example shown in FIG. 4B) can be treated using a medical device 18 (as shown in FIG. 6) having both a proximal stenosis treatment longitudinal zone 19 and a distal stenosis treatment longitudinal zone 20 with either the proximal stenosis treatment longitudinal zone 19 or the distal stenosis treatment longitudinal zone 20 being deployed first, or with both being deployed concurrently, according to clinician and procedural preference.

In some embodiments, a bioresorbable endovascular medical device can be used for placement into a diseased blood vessel that has a dilated portion adjacent a narrowed portion. The medical device can include a generally tubular structure defining a longitudinal axis and a device lumen, the generally tubular structure having at least first and second parts spaced apart along the longitudinal axis, the at least first and second parts being discreet and yet integrally connected to one another and made of substantially the same material, the generally tubular structure configured for placement in a diseased blood vessel that has a dilated portion adjacent a narrowed portion, the blood vessel having a pre-disease healthy cross-sectional area that is smaller than a cross-sectional area of the dilated portion and larger than a cross-sectional area of the narrowed portion. The first part of the generally tubular structure can include a plurality of rings, each ring made up of a plurality of struts and each ring spaced apart from any adjacent ring of the plurality of rings along the longitudinal axis, and can include at least one connecting strut to connect adjacent rings of the plurality of rings, wherein when placed into the narrowed portion of the diseased blood vessel, the first part of the generally tubular structure is configured to increase the cross-sectional area of the narrowed portion and maintain an increased cross-sectional area. The second part of the generally tubular structure can include a tubular mesh, wherein when placed into the dilated portion of the diseased blood vessel, the tubular mesh is configured to direct blood flow along the device lumen while decreasing the flow into the dilated portion of the diseased blood vessel outside of the device lumen, the tubular mesh being porous to thereby promote the formation of thrombus within the dilated portion of the diseased blood vessel outside of the device lumen.

In another aspect, the medical device can include a plurality of struts forming Y-junctures that connect the tubular mesh to at least one ring of the plurality of rings. In another aspect, the tubular mesh of the medical device can include a plurality of strands of material woven together. In another aspect, each strand of the plurality of strands of material can be wound helically around the longitudinal axis. In another aspect, the tubular mesh of the medical device can be woven, knitted, extruded, electrospun, welded, etched, and/or laser cut material.

In another aspect, the first strand of the plurality of strands of material woven together can have a first cross-sectional profile taken perpendicular to a length of the first strand along the longitudinal axis that is different from a second cross-sectional profile of a second strand of the plurality of strands taken perpendicular to a length of the second strand along the longitudinal axis. In another aspect, the at least first and second parts of the medical device can be primarily made of bioresorbable synthetic polymers.

In another aspect, the plurality of rings of the medical device can include a first plurality and a second plurality, the first plurality at a distal-most end of the medical device and the second plurality at a proximal-most end of the medical device, wherein the at least one connecting strut comprises a first connecting strut connecting adjacent rings of the first plurality of rings and a second connecting strut connection adjacent rings of the second plurality of rings.

In another aspect, the generally tubular structure of the medical device can include a structural layer.

In another aspect, the generally tubular structure can include a membrane layer adjacent to a structural layer, the membrane layer defining a porosity of an outer surface of the second part of the generally tubular structure.

In another aspect, the vessel lumen of the medical device can include a hemodynamically stable channel through which a substantial portion of the incoming fluid can flow under normal physiological conditions.

In another aspect, the generally tubular structure of the medical device can include a circumferential array of frame members that are oriented along the longitudinal axis of the vessel, to provide structural support in that axis while allowing for radial vessel expansion.

In another aspect, a method of treating an aneurysm and a stenosis that are adjacent one another in a blood vessel is disclosed. The method can include advancing a delivery device with the bioresorbable endovascular medical device of any of the embodiments described herein in a collapsed state on the delivery device to a treatment location in the diseased blood vessel, and can include deploying the bioresorbable endovascular medical device such that the first portion of the generally tubular structure is within a stenosis and the second portion of the generally tubular structure is within an aneurysm.

In another aspect, a bioresorbable endovascular medical device for placement into a diseased blood vessel having an aneurysm adjacent a stenosis is disclosed. The medical device can include a first generally tubular structure configured such that when placed into a stenosis in a diseased blood vessel, the first generally tubular structure serves to open the stenosis and maintain that opening, and can include a second generally tubular structure connected to the first generally tubular structure that, when placed into the aneurysm of the blood vessel, is configured to establish a vessel lumen and separate excess dilated volume from the vessel lumen.

In another aspect, a method of treating an aneurysm and a stenosis that are adjacent one another in a blood vessel is disclosed. The method can include advancing a delivery device with a bioresorbable endovascular medical device in a collapsed state to a treatment location in a diseased blood vessel, and can include deploying the bioresorbable endovascular medical device, the bioresorbable endovascular medical device comprising a generally tubular structure, a first portion of the generally tubular structure being within a stenosis and a second portion of the generally tubular structure being within an aneurysm.

In another aspect, deploying the bioresorbable endovascular medical device can include foreshortening the second portion as the device moves from the collapsed state to an expanded state.

The advantages of the present medical device include, without limitation, the ability to be delivered percutaneously in small, tortuous vascular anatomy, to be secured in short and/or compromised distal and proximal landing zones, to provide stent-like structural support for potentially narrowed and/or pre-treated aneurysm necks, to achieve sufficient flow restriction to induce aneurysm thrombosis and promote the healing process, to establish and maintain an open and hemodynamically stable primary lumen, to provide non-permanent structural support consistent with a therapeutic timeframe, to be bioresorbed after the therapeutic time frame, and to gradually restore pulsatile response of the vessel to normal vasomechanics. The restoration of vessel response to normal vasomechanics is known in the art of tissue engineering to be essential to regeneration of a vessel as aspects such as cellular morphology, orientation, behavior, and interaction with other cells, fluids, extracellular matrix, and tissue are in many ways dependent upon the cyclical physiologic stresses normally encountered. While this is recognized, it has been a challenge to replicate these stresses in vitro and to take full advantage of these stresses in vivo. This device addresses the initial need to provide support to the dilated vessel and then the subsequent need for the vessel to fully experience normal physiological stresses, by being bioresorbed and disappearing, which biodurable materials and existing device constructs do not do.

The descriptions of the invention are provided in the context of the treatment of arterial defects; however, the present invention can be used in other body passages.

Conditional language used herein, such as, among others, “can,” “could,” “might,” “may,” “e.g.,” “for example,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above, but should be determined only by a fair reading of the claims that follow.

Similarly, this method of disclosure, is not to be interpreted as reflecting an intention that any claim require more features than are expressly recited in that claim. Rather, as the following claims reflect, inventive aspects lie in a combination of fewer than all features of any single foregoing disclosed embodiment. Thus, the claims following the Detailed Description are hereby expressly incorporated into this Detailed Description, with each claim standing on its own as a separate embodiment.

We claim:

1. A bioresorbable endovascular medical device for placement into a diseased blood vessel that has a dilated portion adjacent a narrowed portion, the device comprising:
a generally tubular structure defining a longitudinal axis and a device lumen, the generally tubular structure having at least first and second parts spaced apart along the longitudinal axis, the at least first and second parts being discreet and yet integrally connected to one another and made of substantially the same material, the generally tubular structure configured for placement in a diseased blood vessel that has a dilated portion adjacent a narrowed portion, the blood vessel having a pre-disease healthy cross-sectional area that is smaller than a cross-sectional area of the dilated portion and larger than a cross-sectional area of the narrowed portion, the first part of the generally tubular structure comprising:
a plurality of rings, each ring made up of a plurality of struts and each ring spaced apart from any adjacent ring of the plurality of rings along the longitudinal axis; and
at least one connecting strut to connect adjacent rings of the plurality of rings,
wherein when placed into the narrowed portion of the diseased blood vessel, the first part of the generally tubular structure is configured to increase the cross-sectional area of the narrowed portion and maintain an increased cross-sectional area; and
a second part of the generally tubular structure comprising:
a tubular mesh,
wherein when placed into the dilated portion of the diseased blood vessel, the tubular mesh is configured to direct blood flow along the device lumen while decreasing the flow into the dilated portion of the diseased blood vessel outside of the device lumen, the tubular mesh being porous to thereby promote the formation of thrombus within the dilated portion of the diseased blood vessel outside of the device lumen.

2. The medical device of claim 1, further comprising a plurality of struts forming Y-junctures that connect the tubular mesh to at least one ring of the plurality of rings.

3. The medical device of claim 1, wherein the tubular mesh comprises a plurality of strands of material woven together.

4. The medical device of claim 3, wherein each strand of the plurality of strands of material is wound helically around the longitudinal axis.

5. The medical device of claim 1, wherein the tubular mesh comprises a woven, knitted, extruded, electrospun, welded, etched, or laser cut material.

6. The medical device of claim 3, wherein a first strand of the plurality of strands of material has a first cross-sectional profile taken perpendicular to a length of the first strand along the longitudinal axis that is different from a second cross-sectional profile of a second strand of the plurality of strands taken perpendicular to a length of the second strand along the longitudinal axis.

7. The medical device of claim 1, wherein the at least first and second parts are primarily made of bioresorbable synthetic polymers.

8. The medical device of claim 1, wherein the plurality of rings comprises a first plurality and a second plurality, the first plurality at a distal-most end of the medical device and the second plurality at a proximal-most end of the medical device;
wherein the at least one connecting strut comprises a first connecting strut connecting adjacent rings of the first plurality of rings and a second connecting strut connection adjacent rings of the second plurality of rings.

9. The medical device of claim 1, wherein the generally tubular structure comprises a structural layer.

10. The medical device of claim 9, wherein the generally tubular structure further comprises a membrane layer adjacent to the structural layer, the membrane layer defining a porosity of an outer surface of the second part of the generally tubular structure.

11. The medical device of claim 1, wherein the vessel lumen comprises a hemodynamically stable channel through which a substantial portion of the incoming fluid can flow under normal physiological conditions.

12. The medical device of claim 1, wherein the generally tubular structure comprises a circumferential array of frame members that are oriented along the longitudinal axis of the vessel, to provide structural support in that axis while allowing for radial vessel expansion.

13. A method of treating an aneurysm and a stenosis that are adjacent one another in a blood vessel, comprising:
advancing a delivery device with the bioresorbable endovascular medical device of claim 1 in a collapsed state on the delivery device to a treatment location in the diseased blood vessel; and
deploying the bioresorbable endovascular medical device such that the first portion of the generally tubular structure is within a stenosis and the second portion of the generally tubular structure is within an aneurysm.

14. A bioresorbable endovascular medical device for placement into a diseased blood vessel having an aneurysm adjacent a stenosis, the device comprising:
a first generally tubular structure configured such that when placed into a stenosis in a diseased blood vessel, the first generally tubular structure serves to open the stenosis and maintain that opening; and
a second generally tubular structure connected to the first generally tubular structure that, when placed into the aneurysm of the blood vessel, is configured to establish a vessel lumen and separate excess dilated volume from the vessel lumen.

15. A method of treating an aneurysm and a stenosis that are adjacent one another in a blood vessel, comprising:
advancing a delivery device with a bioresorbable endovascular medical device in a collapsed state to a treatment location in a diseased blood vessel;
deploying the bioresorbable endovascular medical device, the bioresorbable endovascular medical device comprising a generally tubular structure, a first portion of the generally tubular structure being within a stenosis and a second portion of the generally tubular structure being within an aneurysm.

16. The method of claim 15, wherein deploying the bioresorbable endovascular medical device further comprises foreshortening the second portion as the device moves from the collapsed state to an expanded state.
